(19) United States
(12) Patent Application Publication
(10) Pub. No.: US 2007/0009591 A1
(43) Pub. Date: Jan. 11, 2007

(54) ACE inhibitor formulation

(76) Inventors: Jay S. Trivedi, Skokie, IL (US); Gaetan Marcoux, Seymour, IN (US)

Correspondence Address: HARNESS, DICKEY, & PIERCE, P.L.C, 7700 BONHOMME, STE 400, ST. LOUIS, MO, 63105, US

(21) Appl. No.: 11/176,565

(22) Filed: Jul. 7, 2005

(51) Int. Cl. A61K 9/20; B27N 3/00

(52) U.S. Cl. 424/464; 264/109

(57) ABSTRACT

A process for preparing pharmaceutical micro-tablets comprises (a) preparing a tableting mix that comprises an ACE inhibitor, for example moexipril hydrochloride, and excipient ingredients that comprise one or more lubricants; and (b) compressing the tableting mix in a tablet press, to form micro-tablets having an average uncoated weight of about 1 to about 40 mg; wherein the process employs means for promoting release of the mirco-tablets from the tablet press, said release promoting means being unnecessary for otherwise similar standard tablets having an average uncoated weight greater than about 50 mg. A plurality of micro-tablets thus prepared, collectively comprising a therapeutically effective amount of the ACE inhibitor, can be filled into a pharmaceutically acceptable capsule shell to provide a dosage form.

### FIELD OF THE INVENTION

The present invention relates to pharmaceutical compositions for delivery of an angiotensin converting enzyme (ACE) inhibitor to a subject in need thereof, for example as an antihypertensive, and to a process for preparing such a composition.

### BACKGROUND OF THE INVENTION

ACE inhibitors are a widely used class of drugs useful in treatment of hypertension in human and non-human animals. Formulations of ACE inhibitors for oral administration to human patients include standard tablet and capsule formulations. For example, the ACE inhibitor moexipril hydrochloride (Univasc® tablets of Schwarz Pharma, Inc.) is available by prescription in a coated tablet formulation containing 7.5 mg or 15 mg moexipril hydrochloride as active agent. The tablet comprises a core containing, in addition to the active agent, lactose, magnesium oxide, crospovidone, magnesium stearate and gelatin; and surrounded by a film coating containing hypromellose (also known as hydroxypropylmethylcellulose or HPMC), hydroxypropylcellulose, polyethylene glycol (PEG) 6000, magnesium stearate, titanium dioxide and ferric oxide. Product information for Univasc® tablets can be found in literature published by the manufacturer and in compilations such as, for example, Physician's Drug Reference, 59th ed. (2005), pp. 3090-3092 (Montvale, N.J.: Thomson PDR), incorporated in pertinent part herein by reference.

It is desirable to provide alternative formulations of drugs, for example to adapt a dosage form to the particular needs of certain subjects, to clearly differentiate a drug product for reduced risk of pharmacist or patient error, to provide a more convenient dosage form, improve ease of swallowing or otherwise enhance patient compliance, etc. However, it is not always easy to do this without making substantial changes in the formulation and thereby incurring the possibility of changes in bioavailability of the drug. Especially where a drug has a well established efficacy and safety profile, major reformulation is generally to be avoided, as large-scale and expensive clinical trials may be necessary to confirm that bioavailability, and therefore efficacy and safety, have not been substantially affected.

### SUMMARY OF THE INVENTION

There is now provided a process for preparing pharmaceutical tablets, the process comprising (a) preparing a tableting mix that comprises an ACE inhibitor and excipient ingredients that comprise one or more lubricants; and (b) compressing the tableting mix in a tablet press, to form tablets having an average uncoated weight of about 1 to about 40 mg; wherein the process employs means for promoting release of the tablets from the tablet press, said release promoting means being unnecessary for otherwise similar tablets having an average uncoated weight greater than about 50 mg.

In one embodiment, the process further comprises filling a plurality of the tablets into a pharmaceutically acceptable capsule shell to provide a dosage form that can be used to administer a therapeutically effective amount of the ACE inhibitor orally to a subject.

There is further provided a pharmaceutical composition comprising a pharmaceutically acceptable capsule shell having enclosed therewithin a plurality of tablets that collectively comprise (a) a therapeutically effective dosage amount of an ACE inhibitor and (b) excipient ingredients that comprise one or more lubricants; the tablets having an uncoated weight of about 1 to about 40 mg, and the lubricant(s) being present in an increased amount by comparison with standard tablets having an average uncoated weight greater than about 50 mg.

A composition comprising a plurality of tablets within a capsule in accordance with the invention is known as a “micro-tablet composition”. A “micro-tablet” herein refers to one of such a plurality of tablets, typically having an uncoated weight of about 1 to about 40 mg, whether enclosed in a capsule shell or not.

There is still further provided a method for treatment of hypertension in a subject, the method comprising orally administering to the subject at a therapeutically effective dosage frequency one to a plurality of micro-tablets comprising an ACE inhibitor as described above. In one embodiment, at least one capsule having therewithin a plurality of the micro-tablets is administered to the subject.

### DETAILED DESCRIPTION

The present invention arises in part from an unexpected finding that, when using a tableting mix adapted for preparation of a standard tablet formulation of an ACE inhibitor to prepare a plurality of smaller micro-tablets, the resulting micro-tablets failed to release from tablet compressing equipment as cleanly or efficiently as standard tablets prepared from the same mix.

According to the invention, a process for preparing micro-tablets having an average uncoated weight of about 1 to about 40 mg as described herein incorporates means for promoting release of the micro-tablets from a tablet press. The release promoting means are unnecessary for otherwise similar tablets having an average uncoated weight greater than about 50 mg.

Any release promoting means can be used, including without limitation modifications to the tablet press, more particularly to the die and/or punch assemblies, and/or modifications to the tableting mix submitted to the tablet press. One of skill in the art will recognize that some modifications will be more practical, more economical and/or more effective than others, depending on the particular ACE inhibitor and excipient ingredients used, on the design and operating parameters of the tablet press, and other factors. In general, release promoting means operate by reducing adherence of the tableting mix, upon compression thereof, to the die and/or punch of the tablet press.

Suitable release promoting means can illustratively be selected from the following:
(1) modification of the internal shape of the die (and consequently the shape of the resulting tablet), for example to reduce or minimize surface area to volume ratio;
(2) modification, including for example reduction or avoidance, of internal topographical features of the die and/or punch (and consequently topographical features of the resulting tablet) where adherence is more troublesome, for example embossed features;
(3) modification of internal surfaces of the die and/or punch to reduce adherence, for example by provision of an anti-adherent coating such as a polytetrafluoroethylene (e.g., Teflon®) coating;
(4) modification of the tableting mix formula, for example to replace or reduce one or more excipient ingredients such as certain diluents and binders that can promote adherence by producing a “sticky” blend, or to add or increase amount of one or more ingredients that reduce adherence, such as lubricants;
(5) modification of the procedure used to prepare the tableting mix, for example making changes to a granulation procedure that result in more thorough or more intimate mixing of ingredients, or modifying particle size range, e.g., using larger particle sizes, of particular ingredients, to reduce adherence;
   and means equivalent to any of the above.

In one embodiment, the release promoting means comprises surface-modification of the die and/or punch of the tablet press.

In another embodiment, the release promoting means comprises increasing the amount of one or more lubricants in the tableting mix. It has been found that by increasing the amount of one or more lubricants in the mix, clean and efficient release of the micro-tablets from the compressing equipment can be restored.

Those of skill in the art will recognize that if too much lubricant is added to a tableting mix, the resulting tablets can be slow to disintegrate and/or dissolve, and/or have other deficiencies. Reduced disintegration rate or dissolution rate can lead to changes in bioavailability of an active ingredient of the tablets, as measured for example by pharmacokinetic (PK) parameters including Cₘₐₓ, Tₘₐₓ and AUC. According to one embodiment of the present invention, the amount of lubricant in the mix is sufficient to provide clean, efficient release of micro-tablets but not sufficient to retard disintegration and/or dissolution rate of the micro-tablets. Efficiency of tablet release is very important in a commercial manufacturing setting, because it can be a rate-controlling step in the manufacturing process.

The term “ACE inhibitor” herein refers to a compound having inhibitory activity on angiotensin converting enzyme, including prodrugs and salts, or a mixture of two or more of such compounds.

In one embodiment, the ACE inhibitor comprises a compound of formula (I): 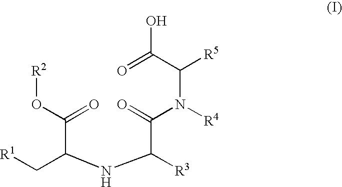
or an isomer or tautomer thereof, or a pharmaceutically acceptable salt of such a compound, isomer or tautomer, where R¹ and R² are independently C₁₋₄ alkyl, phenyl or benzyl; R³ is C₁₋₄ alkyl, amino(C₁₋₄ alkyl), or joined in a ring system with R⁴; R⁴ is a carbocyclic or fused carbocyclic group or is joined in a ring system with R³; and R⁵ is H or is joined in a ring system with R⁴; wherein a ring system if present joining R³ and R⁴ is an optionally substituted seven-member ring, and a ring system if present joining R⁴ and R⁵ is an optionally substituted five- or six-member ring.

Examples of ACE inhibitors of formula (I) include without limitation benazepril, delapril, enalapril, imidapril, lisinopril, moexipril, perindopril, quinapril, ramipril, spirapril, temocapril, trandolapril and pharmaceutically acceptable salts thereof. ACE inhibitors not embraced by formula (I) but useful according to the present invention include without limitation alacepril, captopril, ceronapril, cilazapril, fosinopril, moveltipril, sampatrilat and pharmaceutically acceptable salts thereof.

In a more particular embodiment, the ACE inhibitor comprises a compound of formula (II): 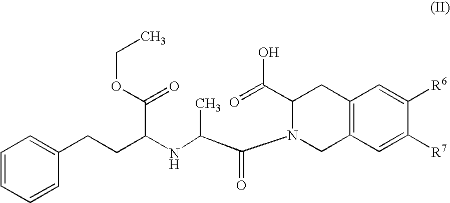
or an isomer or tautomer thereof, or a pharmaceutically acceptable salt of such a compound, isomer or tautomer, where R⁶ and R⁷ are independently H, hydroxy, C₁₋₄ alkyl, C₁₋₄ alkoxy, C₁₋₄ alkylthio, C₁₋₄ alkylsulfinyl or C₁₋₄ alkylsulfonyl groups or R⁶ and R⁷ together form a methylenedioxy group. Examples of ACE inhibitors of formula (II) include without limitation moexipril (where R⁶ and R⁷ are each methoxy), quinapril (where R⁶ and R⁷ are each H) and pharmaceutically acceptable salts thereof.

Pharmaceutically acceptable salts of ACE inhibitors include without limitation hydrochloride, hydrobromide, hydroiodide, sulfate, phosphate, nitrate, benzoate, acetate, fumarate, malonate, succinate, malate, maleate, citrate, tartrate, methanesulfonate and 1,2-ethanedisulfonate salts. Illustratively, the ACE inhibitor comprises moexipril hydrochloride.

The ACE inhibitor is not limited herein to any particular solid state form thereof. For example, an ACE inhibitory compound or its salt can be present in one or more amorphous and/or crystalline forms.

Optionally, a composition of the invention further comprises a drug other than an ACE inhibitor, for example a second drug having cardiovascular or antihypertensive benefits, such as a diuretic. Illustratively, the composition can comprise an ACE inhibitor, for example any ACE inhibitor mentioned above, and a therapeutically effective amount of hydrochlorothiazide.

A “therapeutically effective amount” of an ACE inhibitor or other drug herein is an amount in a single dose that, when the dose is administered repeatedly to a patient according to a regimen, is effective in treatment of hypertension or other cardiovascular condition in the patient. Frequency of administration according to such a regimen can be about four times a day to about once a week, but is typically about twice to about once a day. The dose should not exceed a maximum dose that can be tolerated by the patient without adverse side effects. Illustratively, in the case of moexipril hydrochloride, a therapeutically effective daily dose for most patients is in a range of about 1 mg to about 30 mg, more particularly about 3 mg to about 30 mg, for example 3.75 mg, 7.5 mg, 15 mg or 30 mg, administered in one or two divided doses. Where hydrochlorothiazide is included together with the ACE inhibitor, a therapeutically effective daily dose of hydrochlorothiazide for most patients is in a range of about 5 mg to about 50 mg, for example 12.5 mg or 25 mg.

The active ingredient (an ACE inhibitor, optionally together with a second drug such as hydrochlorothiazide) is formulated in micro-tablets together with excipient ingredients that act, inter alia, as a carrier for the active ingredient. The excipient ingredients include at least one lubricant and can further include one or more materials selected from diluents, disintegrants, binders, pH modifying agents, coloring agents and antioxidants.

The micro-tablets can comprise one or more pharmaceutically acceptable diluents as excipients. Suitable diluents illustratively include, either individually or in combination, lactose, including anhydrous lactose and lactose monohydrate; mannitol; sorbitol; xylitol; dextrose, including dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; carbonates and bicarbonates of calcium, magnesium and sodium; magnesium oxide (light or heavy); glycine; bentonite; polyvinylpyrrolidone (also known as povidone or PVP); and the like. Such diluents typically constitute in total about 5% to about 99%, for example about 10% to about 85% or about 20% to about 80%, by weight of the composition. The diluent or diluents should be selected to exhibit suitable flow properties and compressibility.

The micro-tablets can comprise one or more pharmaceutically acceptable disintegrants as excipients. Suitable disintegrants illustratively include, either individually or in combination, starches, such as starch glycolate (e.g., Explotab™ of PenWest); clays (e.g., Veegum™ HV); cellulose-based disintegrants such as purified cellulose, microcrystalline cellulose, methylcellulose, carmellose, carmellose sodium and croscarmellose sodium; alginates; crospovidone; and gums such as agar, guar, locust bean, karaya, pectin and tragacanth gums. Such disintegrants typically constitute in total about 0.2% to about 30%, for example about 0.2% to about 10% or about 0.2% to about 5%, by weight of the composition.

The micro-tablets can comprise one or more pharmaceutically acceptable binders as excipients. Suitable binders illustratively include, either individually or in combination, acacia; tragacanth; sucrose; gelatin; glucose; celluloses including, but not limited to, methylcellulose, carmellose sodium, HPMC, hydroxypropylcellulose and ethylcellulose; alginic acid and salts thereof; magnesium aluminum silicate; PEG; guar gum; polysaccharide acids; bentonites; povidone, for example povidone K-15, K-30 and K-29/32; and polymethacrylates. Such binders typically constitute in total about 0.5% to about 25%, for example about 0.75% to about 15% or about 1% to about 10%, by weight of the composition.

The micro-tablets optionally comprise one or more pharmaceutically acceptable wetting agents as excipients. Non-limiting examples of surfactants that can be used as wetting agents include quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example polyoxyethylene (20) cetostearyl ether; polyoxyethylene fatty acid esters, for example polyoxyethylene (40) stearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monostearate; sorbitan esters, for example sorbitan monolaurate, sorbitan monooleate, sorbitan monopalmitate and sorbitan monostearate; tyloxapol; and mixtures thereof. One or more wetting agents, if present, can constitute in total about 0.25% to about 15%, for example about 0.4% to about 10% or about 0.5% to about 5%, by weight of the composition.

Other excipients such as pH modifying agents (e.g., buffering agents), coloring agents (e.g., titanium dioxide, ferric oxide, etc.), stabilizers and antioxidants are known in the pharmaceutical art and one or more of such excipients can optionally be present in compositions of the present invention.

As noted above, micro-tablets of the invention comprise at least one lubricant. Lubricants, which can also act as glidants and/or anti-adherents, provide a number of benefits, including without limitation reduction of friction between a tableting mix and tableting equipment, reduction of sticking of the tableting mix to a compression die and/or tablet punch (and consequent improvement of tablet release), and reduction of static.

Selection of lubricant(s) is not narrowly critical. Suitable lubricants illustratively include, either individually or in combination, glyceryl behenate (e.g., Compritol™ 888); stearic acid and salts thereof, including magnesium, calcium and sodium stearates; hydrogenated vegetable oils (e.g., Sterotex™); colloidal silica; talc, for example micronized talc; waxes; boric acid; sodium benzoate; sodium acetate; sodium fumarate; sodium chloride; DL-leucine; PEG (e.g., PEG 4000 and PEG 6000); sodium oleate; sodium stearyl fumarate; sodium lauryl sulfate; and magnesium lauryl sulfate.

In one embodiment, the at least one lubricant comprises magnesium stearate.

Although the selection of lubricant(s) is not critical, the amount of such lubricant(s) is, in one embodiment of the invention, critical to ensuring an efficient tablet manufacturing process. Surprisingly, an amount of lubricant that is adequate for efficient processing of a particular tableting mix to prepare conventional tablets is not necessarily adequate where the same tableting mix is used to prepare micro-tablets.

In one embodiment, one or more lubricants are present in the micro-tablet formulation of the invention in an increased amount by comparison with a standard tablet formulation containing the same dosage amount of the ACE inhibitor. In another embodiment, one or more lubricants are present in the micro-tablet formulation of the invention in an amount substantially greater than a minimum amount required for efficient production of a standard tablet formulation prepared from a tableting mix that is similar but for the amount of the lubricant(s). In yet another embodiment, a micro-tablet composition of the invention is derived from an existing standard tablet composition that contains a lubricant, by reformulation of the tableting mix to increase the amount of the lubricant. According to this embodiment, the amount of the lubricant is increased by at least about 5%, for example by at least about 10%, but to a degree no greater than is consistent with acceptable disintegration and dissolution rate of the micro-tablet. In general a good result will be obtained by increasing the amount of the lubricant by no more than about 50%, in most cases by no more than about 30%, or even no more than about 20%. Illustratively, an increase of the order of about 15%, for example about 10% to about 20%, or about 12% to about 18%, in the amount of the lubricant can be sufficient.

One of skill in the art will recognize that a suitable absolute amount of lubricant to be used depends in part on the particular lubricant selected, and will henceforward be able to identify a suitable amount of increase in lubricant amount without undue experimentation, based on information provided herein. As further guidance, the following illustrative amounts are for micro-tablets having magnesium stearate as a lubricant.

For a standard single-dose tablet having an uncoated tablet weight of at least about 50 mg, typically about 50 to about 400 mg, a suitable amount of magnesium stearate can be, for example, about 0.75% by weight of the uncoated tablet. Where, instead, a plurality of about 5 to about 50 micro-tablets, each having an uncoated micro-tablet weight of about 5 to about 20 mg, are to deliver a single dose of the drug, the amount of magnesium stearate should be increased to at least about 0.79% (an increase of about 5%), more typically to at least about 0.82% (an increase of about 10%).

In one embodiment, a micro-tablet composition of the invention comprises about 0.8% to about 1.2%, for example about 0.85% to about 1.0%, magnesium stearate.

It will be understood that such percentages are averages, and that individual micro-tablets, or individual capsules containing a plurality of micro-tablets, in a batch (e.g., a manufacturing lot) can deviate from the ranges given without excluding the batch, having an average within these ranges, from the scope of the invention.

Examples of excipient ingredients provided herein are not intended to be exhaustive. Other excipients can be selected by those of skill in the art without departing from the present invention. A more extensive list of excipients can be found, for example, in U.S. Patent Application Publication No. 2003/0104048, incorporated by reference in its entirety herein.

The tableting mix is prepared according to any known procedure and, with the exception of a higher lubricant amount, can be prepared in exactly the same way as for making standard single-dose tablets. Typically a granulation (either wet or dry granulation) procedure is used in preparing the tableting mix.

Compression of the tableting mix to form micro-tablets can be performed using any suitable tableting press fitted with dies of the desired dimensions. One of skill in the art will readily establish suitable compression force and other settings to result in a smooth tableting operation. In general it will be found desirable not to exceed about 15 kN compression force. Illustratively, a compression force of about 4 to about 13.5 kN, for example about 4 to about 8 kN, applied via the punch can be suitable.

Micro-tablets can be prepared in any convenient shape but are typically round with more or less flat upper and lower surfaces. A round micro-tablet having an uncoated weight of about 5 to about 20 mg can illustratively be made at a diameter of about 1.5 to about 3 mm, for example about 2.25 mm.

The micro-tablets can be coated, for example with an immediate-release, delayed-release or enteric coating, or with a non-release-modifying (e.g., a moisture barrier) coating, or uncoated. In one embodiment a polymer-based film coating is present around each micro-tablet. Suitable polymers can include one or more of HPMC; hydroxypropylcellulose; PEG, for example PEG 6000; acrylic polymers and the like. The film coating can additionally comprise other ingredients, such as plasticizers, colorants, for example titanium dioxide and ferric oxide, etc. A coating on a micro-tablet typically increases its weight by about 2% to about 5%, for example about 3.5%. According to the process of the invention, coating is an optional step following compression of the micro-tablets.

Micro-tablets prepared as described above can be the end-product of the process. They are suitable as such for oral administration to a subject for whom an ACE inhibitor is indicated. Typically, a plurality of micro-tablets, for example about 5 to about 50, provide a single therapeutically effective dose of the ACE inhibitor and optionally of a second drug such as hydrochlorothiazide.

In one embodiment, however, the process further comprises filling a plurality of the coated or uncoated micro-tablets into a pharmaceutically acceptable capsule shell. The resulting capsule typically represents a single dosage unit, and is itself an embodiment of the present invention. Any suitable capsule material can be used, including without limitation gelatin, HPMC and combinations thereof. A capsule size can be selected appropriate to the number and size of micro-tablets to be placed in each capsule. Where the term “filling” is used herein with respect to placement of micro-tablets in a capsule, it will be understood that the internal volume of the capsule is not necessarily fully occupied by the micro-tablets.

Capsule filling requires a desired number of micro-tablets per capsule to be accurately and reproducibly counted. Equipment with suitable controls for this task is commercially available.

The number of micro-tablets per capsule is not narrowly critical but in most cases a number of about 5 to about 50 will be found suitable. Illustratively, in the case of moexipril hydrochloride, a 7.5 mg dose can be delivered in 9 micro-tablets and a 15 mg dose in 18 micro-tablets, in each case provided within a single capsule; however, these numbers can be varied if desired. In one embodiment, moexipril hydrochloride is the sole active ingredient in the micro-tablets. However, optionally and illustratively, a 7.5 mg dose of moexipril hydrochloride can be accompanied by a 12.5 mg dose of hydrochlorothiazide, or a 15 mg dose of moexipril hydrochloride can be accompanied by a 12.5 mg or 25 mg dose of hydrochlorothiazide, in the plurality of micro-tablets providing such doses.

Typically, each micro-tablet has a total uncoated weight of about 5 to about 20 mg, although smaller or greater weights can be useful in certain situations. It will be understood that there is generally an inverse relationship between micro-tablet number and micro-tablet weight. At weights above about 20 mg, the benefits of a micro-tablet formulation of the invention over a standard tablet formulation become more limited, and at weights below about 5 mg ease of manufacture diminishes. Illustratively, micro-tablets can have an uncoated weight of about 7 to about 15 mg, for example about 10 mg.

A particular benefit of the present process, by comparison with formulating the same drug as a standard single-dose tablet, is that just one kind of micro-tablet can serve to prepare numerous dosage strengths with a high degree of precision, simply by controlling the number of micro-tablets placed in a capsule. This can be economically advantageous, for example in minimizing “down time” while tableting equipment is converted from one dosage strength to another, and in reducing inventory of tableted product.

A method for treatment of hypertension in a subject comprises orally administering to the subject at a therapeutically effective dosage frequency one to a plurality of micro-tablets of the invention. In one embodiment, at least one capsule containing a plurality of the micro-tablets is administered. As indicated above, typical dosage frequencies are from about four times a day to about once a week, most typically about twice to about once per day.

If desired, a capsule can be opened and individual micro-tablets counted out as a convenient way to reduce or customize dosage amount. Micro-tablets can be distributed in an edible vehicle to ease administration for patients having difficulty in swallowing whole capsules or standard single-dose tablets.

The subject can be human or non-human. Examples of non-human subjects for which the present method can be useful include companion animals (e.g., dogs and cats), horses, captive animals, for example in zoos, including non-human primates, farm animals, etc.

The words “comprise”, “comprises”, and “comprising” herein are to be interpreted inclusively rather than exclusively.

The invention is further illustrated but not limited by the following Examples.

### EXAMPLES

### Example 1

In an attempt to prepare micro-tablets, each containing 0.833 mg moexipril hydrochloride, an amount calculated to provide a dose of 7.5 mg in 9 micro-tablets or 15 mg in 18 micro-tablets, a standard wet granulation process is used to prepare a tableting mix having the following ingredients per micro-tablet:

| | |
|---|---|
| moexipril hydrochloride | 0.833 mg |
| lactose monohydrate | 7.849 mg |
| crospovidone (Polyplasdone ™ XL-10) | 0.833 mg |
| magnesium oxide (light) | 0.186 mg |
| magnesium stearate | 0.078 mg |
| gelatin | 0.075 mg |

The tableting mix is fed to a manufacturing-scale tableting press, which has been successfully used to prepare standard single-dose 7.5 mg and 15 mg moexipril hydrochloride tablets from a similar tableting mix. The press is modified for preparation of 2.25 mm diameter round micro-tablets. It is found that the micro-tablets tend to stick to the press, resulting in operation of the press being stopped for cleaning every few minutes.

### Example 2

In another attempt to prepare micro-tablets, each containing 0.833 mg moexipril hydrochloride as above, a tableting mix having the following ingredients per micro-tablet is prepared as in Example 1:

| | |
|---|---|
| moexipril hydrochloride | 0.833 mg |
| lactose monohydrate | 7.849 mg |
| crospovidone (Polyplasdone ™ XL-10) | 0.833 mg |
| magnesium oxide (light) | 0.186 mg |
| magnesium stearate | 0.089 mg |
| gelatin | 0.075 mg |

It will be noted that the composition of the tableting mix is the same as in Example 1, except that the amount of magnesium stearate is increased from 0.078 to 0.089 mg (a 14% increase). The tableting mix is fed to the tableting press as in Example 1. Micro-tablets are successfully prepared without unacceptable sticking to the press or failure to eject. The micro-tablets, 2.25 mm in diameter, have an average uncoated weight of 10.01 mg.

### Example 3

Micro-tablets prepared as in Example 2 are coated with 0.37 mg white Opadry™ film-forming polymer coating as a moisture barrier. The average finished (coated) weight of the micro-tablets is 10.38 mg.

The coated micro-tablets are filled into gelatin or HPMC capsules which are then sealed. Nine micro-tablets are filled into a capsule to prepare a 7.5 mg moexipril hydrochloride dosage form, and 18 micro-tablets are filled into a capsule to prepare a 15 mg moexipril hydrochloride dosage form.

### Example 4

Gelatin or HPMC capsules filled as in Example 3 to prepare 7.5 mg moexipril hydrochloride dosage forms are subjected to an in vitro dissolution test. Both gelatin and HPMC capsules with their micro-tablet contents exhibit>75% dissolution in 30 minutes, a dissolution performance similar to standard 7.5 mg moexipril hydrochloride tablets.

What is claimed is:

1. A process for preparing pharmaceutical micro-tablets, the process comprising (a) preparing a tableting mix that comprises an ACE inhibitor and excipient ingredients that comprise one or more lubricants; and (b) compressing the tableting mix in a tablet press, to form micro-tablets having an average uncoated weight of about 1 to about 40 mg; wherein the process employs means for promoting release of the micro-tablets from the tablet press, said release promoting means being unnecessary for otherwise similar standard tablets having an average uncoated weight greater than about 50 mg.

2. The process of claim 1, wherein the release promoting means comprises a tablet press having a surface-modified die and/or punch to reduce adherence of the compressed tableting mix thereto.

3. The process of claim 1, wherein the release promoting means comprises a tableting mix having an excipient ingredient composition modified to reduce adherence of the mix to the tablet press upon compression therein.

4. The process of claim 3, wherein modification of the excipient ingredient composition comprises increase in the amount of said one or more lubricants by comparison with a standard tableting mix that provides efficient release of standard tablets having an average uncoated weight greater than about 50 mg.

5. The process of claim 4, wherein the amount of said lubricant(s) is increased by about 5% to about 50% by comparison with said standard tableting mix.

6. The process of claim 4, wherein the amount of said lubricant(s) is increased by about 10% to about 20% by comparison with said standard tableting mix.

7. The process of claim 4, wherein the amount of said lubricant(s) is increased by about 12% to about 18% by comparison with said standard tableting mix.

8. The process of claim 4, wherein modification of the excipient ingredient composition consists essentially of said increase in the amount of said one or more lubricants.

9. The process of claim 4, wherein said lubricant(s) are selected from the group consisting of glyceryl behenate, stearic acid, magnesium stearate, calcium stearate, sodium stearate, hydrogenated vegetable oils, colloidal silica, talc, waxes, boric acid, sodium benzoate, sodium acetate, sodium fumarate, sodium chloride, DL-leucine, PEG, sodium oleate, sodium stearyl fumarate, sodium lauryl sulfate, magnesium lauryl sulfate and mixtures thereof.

10. The process of claim 4, wherein said lubricant(s) comprise magnesium stearate.

11. The process of claim 10, wherein the tableting mix comprises about 0.8% to about 1.2% magnesium stearate.

12. The process of claim 10, wherein the tableting mix comprises about 0.85% to about 1.0% magnesium stearate.

13. The process of claim 1, wherein the ACE inhibitor comprises a compound having the formula: 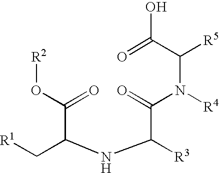
or an isomer or tautomer thereof, or a pharmaceutically acceptable salt of such a compound, isomer or tautomer, where R¹ and R² are independently C₁₋₄ alkyl, phenyl or benzyl; R³ is C₁₋₄ alkyl, amino(C₁₋₄ alkyl), or joined in a ring system with R⁴; R⁴ is a carbocyclic or fused carbocyclic group or is joined in a ring system with R³; and R⁵ is H or is joined in a ring system with R⁴; wherein a ring system if present joining R³ and R⁴ is an optionally substituted seven-member ring, and a ring system if present joining R⁴ and R⁵ is an optionally substituted five- or six-member ring.

14. The process of claim 1, wherein the ACE inhibitor comprises a compound selected from the group consisting of benazepril, delapril, enalapril, imidapril, lisinopril, moexipril, perindopril, quinapril, ramipril, spirapril, temocapril, trandolapril, alacepril, captopril, ceronapril, cilazapril, fosinopril, moveltipril, sampatrilat and pharmaceutically acceptable salts thereof.

15. The process of claim 1, wherein the ACE inhibitor comprises a compound having the formula: 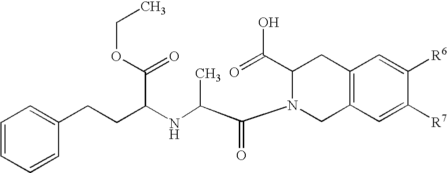
or an isomer or tautomer thereof, or a pharmaceutically acceptable salt of such a compound, isomer or tautomer, where R⁶ and R⁷ are independently H, hydroxy, C₁₋₄ alkyl, C₁₋₄ alkoxy, C₁₋₄ alkylthio, C₁₋₄ alkylsulfinyl or C₁₋₄ alkylsulfonyl groups or R⁶ and R⁷ together form a methylenedioxy group.

16. The process of claim 1, wherein the ACE inhibitor comprises moexipril or a pharmaceutically acceptable salt thereof.

17. The process of claim 1, wherein the ACE inhibitor comprises moexipril hydrochloride.

18. The process of claim 1, wherein the tableting mix further comprises a therapeutically effective amount of hydrochlorothiazide.

19. The process of claim 1, wherein said excipients further comprise one or more pharmaceutically acceptable diluents.

20. The process of claim 1, wherein said excipients further comprise one or more pharmaceutically acceptable disintegrants.

21. The process of claim 1, wherein said excipients further comprise one or more pharmaceutically acceptable binders.

22. The process of claim 1, wherein the tableting mix is prepared by a procedure that comprises granulating the ACE inhibitor and at least part of the excipient ingredients.

23. The process of claim 22, wherein said procedure comprises wet granulation.

24. The process of claim 1, wherein said micro-tablets have an average weight of about 5 to about 20 mg.

25. The process of claim 1, wherein said micro-tablets are round and have a diameter of about 1.5 to about 3 mm.

26. The process of claim 1, further comprising coating the micro-tablets.

27. The process of claim 26, wherein the coating provides a moisture barrier layer around each micro-tablet.

28. The process of claim 1, further comprising filling a plurality of the micro-tablets into a pharmaceutically acceptable capsule shell.

29. The process of claim 28, wherein said plurality of micro-tablets collectively comprise a therapeutically effective dosage amount of the ACE inhibitor.

30. The process of claim 28, wherein about 5 to about 50 of the micro-tablets are filled into each capsule shell.

31. The process of claim 28, wherein the capsule shell is formed from a material comprising gelatin, HPMC or a combination thereof.

32. A process for preparing a pharmaceutically acceptable moexipril dosage form, the process comprising (a) preparing a tableting mix that comprises (i) about 1 to about 30 mg moexipril hydrochloride per dosage form and (ii) excipient ingredients that comprise magnesium stearate in an amount of about 0.8% to about 1.2%; (b) compressing the tableting mix in a tablet press, to form micro-tablets having an average uncoated weight of about 5 to about 20 mg; (c) coating the micro-tablets with a moisture barrier layer; and (d) filling about 5 to about 50 of said micro-tablets into a pharmaceutically acceptable capsule shell to provide the dosage form.

33. The process of claim 32, wherein 9 of said micro-tablets, each of about 10 mg in weight, comprising in total 7.5 mg moexipril hydrochloride, are filled into each capsule shell.

34. The process of claim 32, wherein 18 of said micro-tablets, each of about 10 mg in weight, comprising in total 15 mg moexipril hydrochloride, are filled into each capsule shell.

35. A pharmaceutical composition comprising a pharmaceutically acceptable capsule shell having enclosed therewithin a plurality of micro-tablets that collectively comprise (a) a therapeutically effective dosage amount of an ACE inhibitor and (b) excipient ingredients that comprise one or more lubricants; said micro-tablets having an average uncoated weight of about 1 to about 40 mg; and said lubricant(s) being present in an increased amount by comparison with standard tablets having an average uncoated weight greater than about 50 mg.

36. A pharmaceutical composition comprising a pharmaceutically acceptable capsule shell having enclosed therewithin a plurality of coated micro-tablets that collectively comprise (a) about 1 to about 30 mg moexipril hydrochloride, and (b) excipient ingredients that comprise magnesium stearate in an amount of about 0.8% to about 1.2%, said micro-tablets having an average uncoated weight of about 5 to about 20 mg.

37. The composition of claim 36, having 9 of said micro-tablets, each of about 10 mg in weight, per capsule and comprising in total 7.5 mg moexipril hydrochloride.

38. The composition of claim 37, wherein the plurality of micro-tablets further collectively comprise 12.5 mg hydrochlorothiazide.

39. The composition of claim 36, having 18 of said micro-tablets, each of about 10 mg in weight, per capsule and comprising in total 15 mg moexipril hydrochloride.

40. The composition of claim 39, wherein the plurality of micro-tablets further collectively comprise 25 mg hydrochlorothiazide.

41. A method for treatment of hypertension in a subject, the method comprising orally administering to the subject at a therapeutically effective dosage frequency one to a plurality of micro-tablets that collectively comprise (a) a therapeutically effective dosage amount of an ACE inhibitor and (b) excipient ingredients that comprise one or more lubricants; said micro-tablets having an average uncoated weight of about 1 to about 40 mg; and said lubricant(s) being present in an increased amount by comparison with standard tablets having an average uncoated weight greater than about 50 mg.

42. The method of claim 41, wherein at least one capsule having therewithin a plurality of said micro-tablets is administered to the subject.
